(19) United States
(12) Patent Application Publication
(10) Pub. No.: US 2024/0382265 A1
(43) Pub. Date: Nov. 21, 2024

(54) HYBRID LOCALIZATION FOR MINIMALLY INVASIVE SURGERY AND CERVICAL SPINAL REFERENCING, AND METHODS FOR USING THE SAME

(71) Applicant: Medtronic Navigation, Inc., Louisville, CO (US)

(72) Inventors: Justin A. Kemp, Erie, CO (US); Victor D. Snyder, Erie, CO (US); Nikita Pandey, Broomfield, CO (US); Andrew J. Wald, Fort Worth, TX (US); Anisha Parkar, Thornton, CO (US)

(21) Appl. No.: 18/653,392

(22) Filed: May 2, 2024

Related U.S. Application Data
(60) Provisional application No. 63/466,620, filed on May 15, 2023.

(51) Int. Cl. A61B 34/20; A61B 34/30; A61B 90/00

(52) U.S. Cl. CPC A61B 34/20; A61B 34/30; A61B 90/39; A61B 2034/2051; A61B 2034/2055; A61B 2034/2065

(57) ABSTRACT

A method according to at least one embodiment of the present disclosure includes: providing a first localizer relative to a patient anatomy; providing a second localizer in proximity to the first localizer; co-registering the first localizer and the second localizer; determining, based on tracking a pose of the first localizer, first localizer pose information; determining, based on a combination of the first localizer pose information and the co-registration of the first localizer with the second localizer, second localizer pose information; and outputting the second localizer pose information to at least one of a display device and a robotic controller.

### CROSS-REFERENCE TO RELATED APPLICATION

This application claims the benefit of and priority to U.S. Provisional Patent Application No. 63/466,620 filed May 15, 2023, the entire disclosure of which is incorporated by reference herein.

### BACKGROUND

The present disclosure is generally directed to surgeries and surgical procedures, and relates more particularly to localization during surgeries or surgical procedures.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A method according to at least one embodiment of the present disclosure comprises: providing a first localizer relative to a patient anatomy; providing a second localizer in proximity to the first localizer; co-registering the first localizer and the second localizer; determining, based on tracking a pose of the first localizer, first localizer pose information; determining, based on a combination of the first localizer pose information and the co-registration of the first localizer with the second localizer, second localizer pose information; and outputting the second localizer pose information to at least one of a display device and a robotic controller.

Any of the features herein, wherein one or more surgical instruments are navigated based at least partially on the second localizer pose information.

Any of the features herein, wherein the co-registering the first localizer and the second localizer comprises: providing a tracking tool that includes an optical tracker and an electromagnetic tracker in a predetermined pose relative to the optical tracker.

Any of the features herein, wherein the optical tracker comprises a plurality of navigation markers, and wherein the method further comprises: determining a pose of the second localizer relative to the electromagnetic tracker.

Any of the features herein, wherein the tracking tool is provided on at least one of a patient and a patient bed.

Any of the features herein, wherein the co-registering the first localizer and the second localizer comprises: providing an electromagnetic field emitter proximate the patient anatomy; and determining a pose of one or more navigation markers relative to the electromagnetic field emitter.

Any of the features herein, wherein the one or more navigation markers are tracked optically.

Any of the features herein, further comprising: providing an optical camera that comprises an electromagnetic field emitter and that tracks the pose of the first localizer; and determining a pose of the second localizer relative to the electromagnetic field emitter.

A system according to at least one embodiment of the present disclosure comprises: a first localizer; a second localizer positionable proximate the first localizer; a processor; and a memory storing data thereon that, when processed by the processor, cause the processor to: co-register the first localizer and the second localizer; determine, based on tracking a pose of the first localizer, first localizer pose information; determine, based on a combination of the first localizer pose information and the co-registration of the first localizer with the second localizer, second localizer pose information; and output the second localizer pose information to at least one of a display device and a robotic controller.

Any of the features herein, wherein the first localizer is positioned relative to an anatomical element and disposed on at least one of a surgical tool and a bed mount.

Any of the features herein, wherein the second localizer comprises an electromagnetic device.

Any of the features herein, wherein the anatomical element comprises a vertebra, and wherein the second localizer is disposable on the vertebra.

Any of the features herein, wherein the first localizer and the second localizer are co-registered by: identifying an optical tracker and an electromagnetic tracker disposed in a pose relative to the optical tracker.

Any of the features herein, wherein the first localizer and the second localizer are co-registered by: determining a pose of one or more optically tracked navigation markers relative to an electromagnetic field emitter.

Any of the features herein, wherein the first localizer and the second localizer are co-registered by: determining a pose of the second localizer relative to an electromagnetic field emitter; and determining a pose of the electromagnetic field emitter relative to an optical camera that optically tracks the pose of the first localizer.

A system according to at least one embodiment of the present disclosure comprises: a processor; and a memory storing data thereon that, when processed by the processor, cause the processor to: co-register a first localizer and a second localizer, the first localizer positionable to a patient anatomy and the second localizer positionable proximate the first localizer; determine, based on information from an optical camera that tracks a pose of the first localizer, first localizer pose information; determine, based on a combination of the first localizer pose information and the co-registration of the first localizer with the second localizer, second localizer pose information; and output the second localizer pose information to at least one of a display device and a robotic controller.

Any of the features herein, wherein the co-registering the first localizer and the second localizer comprises: identifying a tracking tool that comprises one or more one or more optical navigation markers and an electromagnetic tracker disposed in a pose relative to the one or more optical navigation markers.

Any of the features herein, wherein the co-registering the first localizer and the second localizer comprises: determining a pose of one or more optical navigation markers relative to an electromagnetic field emitter.

Any of the features herein, wherein the co-registering the first localizer and the second localizer comprises: determining a pose of the second localizer relative to an electromagnetic field emitter; and determining a pose of the electromagnetic field emitter relative to the optical camera.

Any of the features herein, wherein the robotic controller navigates one or more surgical instruments based at least partially on the second localizer pose information.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a diagram of aspects of a system according to at least one embodiment of the present disclosure;

FIG. 1B is a diagram of additional aspects of the system according to at least one embodiment of the present disclosure;

FIG. 1C is a diagram of additional aspects of the system according to at least one embodiment of the present disclosure;

FIG. 1D is a diagram of a localizer and an anatomical element according to at least one embodiment of the present disclosure;

FIG. 1E is a diagram of a tracking tool according to at least one embodiment of the present disclosure;

FIG. 2 is a block diagram of additional aspects of the system according to at least one embodiment of the present disclosure;

FIG. 3 is a flowchart according to at least one embodiment of the present disclosure

FIG. 4 is a flowchart according to at least one embodiment of the present disclosure;

FIG. 5 is a flowchart according to at least one embodiment of the present disclosure; and

FIG. 6 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

Spinal navigation can use optical localization. The optical trackers used in optical localization are usually large, and the patient tracker requires a correspondingly large mechanism to attach to the patient anatomy. This tracker can get in the surgeon's way, the tracker's sizable attachment mechanism runs counter to trends toward minimally invasive surgery, and certain anatomical features (e.g., the cervical spine) offer few reasonable attachment points for the attachment mechanisms. A tracker with a smaller profile may beneficially address these issues.

According to at least one embodiment of the present disclosure, a second localizer is incorporated into a spinal navigation system. The second localizer is co-registered to a first localizer. The second localizer's modality enables a patient tracker with favorable properties, especially a smaller tracker. The first localizer tracks optical tools, imagers, etc., while the second localizer tracks patient anatomy. In some embodiments, the second localizer is electromagnetic (e.g., capable of being used by and tracked with electromagnetic systems). In some embodiments, the first and second localizers may be used in navigated spinal fusion procedures, which may include procedures related to the cervical spine.

In some embodiments, the use of the second, smaller localizer may provide a technical solution to issues such as: concerns related to accidental movement of the patient tracker during surgery (e.g., due to bumps, vibrations, etc.), patient concerns about pain associated with the localizer (e.g., percutaneous pinning), and issues associated with limited referencing due to the small size of cervical anatomy.

In some embodiments, the second localizer may be electromagnetic. The electromagnetic nature of the second localizer may permit the second localizer to be much smaller than an optical localizer, to avoid line-of-sight issues associated with an optical localizer, and may enable the second localizer to be in wired or wireless communication with other surgical components.

In some embodiments, the electromagnetic localizer and the optical localizer may be co-registered by using a hybrid tool that is placed proximate the patient (e.g., held on a rigid or articulating arm that is clamped to the patient bed). The marker locations on the hybrid tool may be known in both the localizers' coordinate systems. For example, the sphere (or other navigation marker) post locations may be known in the electromagnetic tracker space due to factory calibration settings. Due to the knowledge of the marker locations in both the electromagnetic and the optical coordinate systems, navigation of surgical tools (e.g., using optical markers) may be enabled while also tracking patient anatomy (e.g., using electromagnetic markers).

In some embodiments, the electromagnetic localizer and the optical localizer may be co-registered by optically tracking an electromagnetic emitter. The electromagnetic emitter might be disposed in a known location relative to optical markers (or vice versa). The optical markers may then be localized relative to the electromagnetic emitter during the electromagnetic emitter calibration process.

In some embodiments, the electromagnetic localizer and the optical localizer may be co-registered by using a camera that includes an electromagnetic emitter. For example, the camera may be placed near the surgical site (e.g., close enough that the electromagnetic emitter in the camera can generate an electromagnetic field that interacts with the electromagnetic localizer), and the pose of the camera may be tracked in an electromagnetic coordinate system based on the location of the electromagnetic emitter.

Embodiments of the present disclosure provide technical solutions to one or more of the problems of (1) tracking patient anatomy and (2) tracking surgical tools.

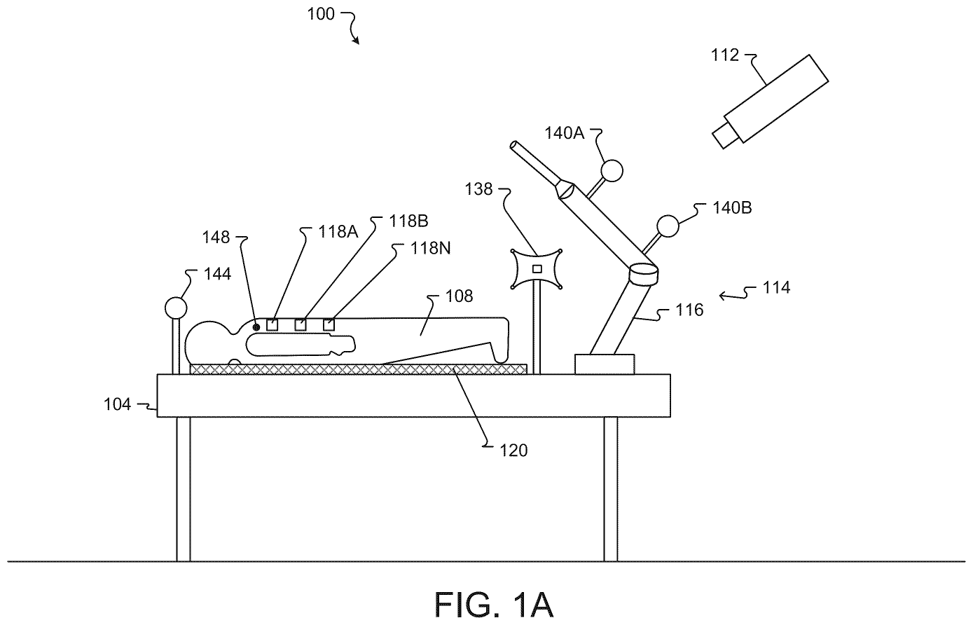

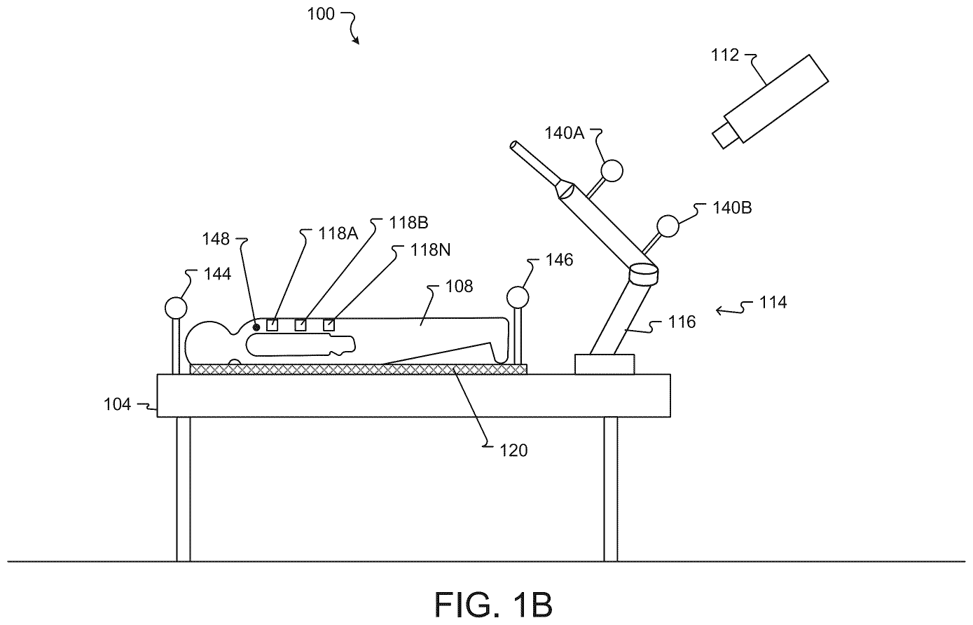

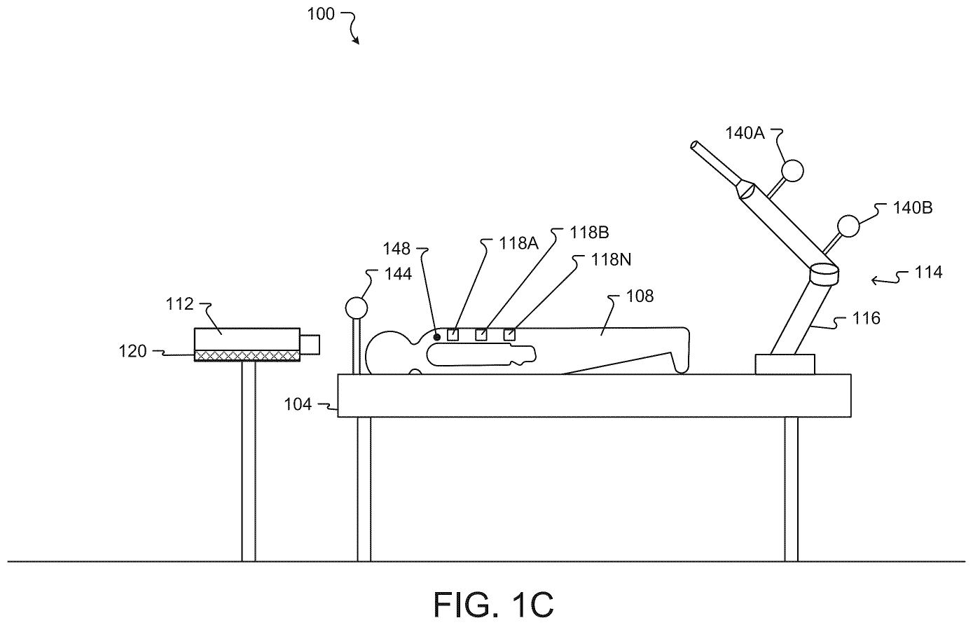

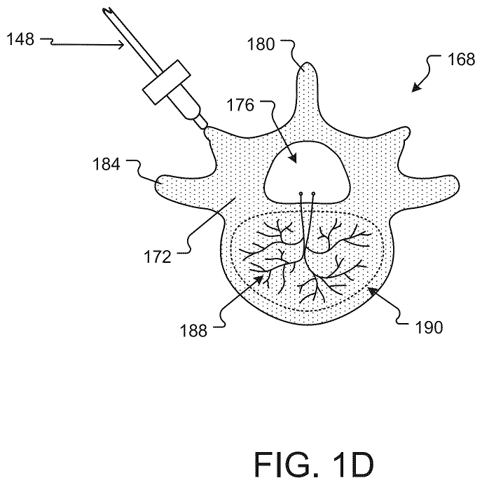

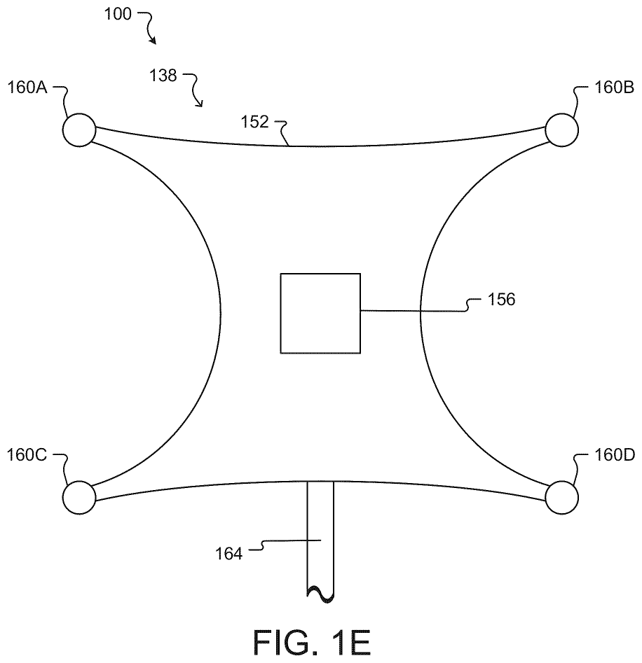

Turning first to FIGS. 1A-1E, aspects of a system 100 according to at least one embodiment of the present disclosure are shown. The system 100 may be used to track and navigate one or more surgical tools; to control, pose, and/or otherwise manipulate a surgical mount system, a surgical arm, and/or surgical tools attached thereto; and/or carry out one or more other aspects of one or more of the methods disclosed herein. The system 100 comprises one or more imaging devices 112, a robot 114 that includes a robotic arm 116, an electromagnetic field emitter 120, a tracking tool 138, navigation markers 140A-140B, a first localizer 144, and a second localizer 148. Systems according to other embodiments of the present disclosure may comprise more or fewer components than the system 100.

The system 100 may include aspects that can be used in or otherwise used to carry out a surgery or surgical procedure. As depicted in FIG. 1A, a patient 108 may be undergoing a surgery or surgical procedure, and the imaging device 112, the robot 114, the robotic arm 116, and the electromagnetic field emitter 120 may be positioned proximate the patient 108. The first localizer 144 may be disposed proximate the patient 108, while the second localizer 148 may be disposed proximate anatomical elements 118A-118N of the patient 108. In one embodiment, the surgery or surgical procedure may be or comprise a spinal fusion of two or more cervical vertebrae together, with the second localizer 148 disposable on or next to a cervical vertebra.

While undergoing the surgery or surgical procedure, the patient 108 may be positioned on a table 104. The table 104 may be any table 104 configured to support a patient during a surgical procedure. The table 104 may include any accessories mounted to or otherwise coupled to the table 104 such as, for example, a bed rail, a bed rail adaptor, an arm rest, an extender, or the like. In some embodiments, the table 104 may comprise a bed mount that enables one or more components to be connected to the table 104. The bed mount may enable, for example, the tracking tool 138, the navigation markers 140A-140B, the first localizer 144, and the like to be attached or connected to the table 104. The table 104 may be stationary or may be operable to maneuver a patient (e.g., the table 104 may be able to move). In some embodiments, the table 104 has two positioning degrees of freedom and one rotational degree of freedom, which allows positioning of the specific anatomy of the patient anywhere in space (within a volume defined by the limits of movement of the table 104). For example, the table 104 can slide forward and backward and from side to side, and can tilt (e.g., around an axis positioned between the head and foot of the table 104 and extending from one side of the table 104 to the other) and/or roll (e.g., around an axis positioned between the two sides of the table 104 and extending from the head of the table 104 to the foot thereof). In other embodiments, the table 104 can bend at one or more areas (which bending may be possible due to, for example, the use of a flexible surface for the table 104, or by physically separating one portion of the table 104 from another portion of the table 104 and moving the two portions independently). In at least some embodiments, the table 104 may be manually moved or manipulated by, for example, a surgeon or other user, or the table 104 may comprise one or more motors, actuators, and/or other mechanisms configured to enable movement and/or manipulation of the table 104 by a processor.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a two-dimensional (2D) image or a three-dimensional (3D) image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient or a feature of a component of the system 100 (e.g., the tracking tool 138, the navigation markers 140A-140B, the first localizer 144, etc.). The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. In some embodiments, the robot 114 may be additionally or alternatively connected to the imaging device 112 and/or to one or more other components of the system 100 (e.g., surgical tools or instruments). The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from a navigation system or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable a processor (or a processor of the robot 114) to determine a precise pose in space of the robotic arm 116 (as well as any object or element held by or secured to the robotic arm 116).

In some embodiments, navigation markers 140A-140B (e.g., reference markers) may be placed on the robot 114 (including, e.g., on the robotic arm 116), the imaging device 112, or any other object in the surgical space. The navigation markers 140A-140B may be tracked optically by a navigation system, such as a navigation system 218 as discussed below, and the results of the tracking may be used by the robot 114 and/or by an operator of the system 100 or any component thereof. In some embodiments, the navigation system can be used to track other components of the system (e.g., the imaging device 112, the tracking tool 138, the first localizer 144, second localizer 148, etc.) and the system can operate without the use of the robot 114 (e.g., with the surgeon manually manipulating the imaging device 112 and/or one or more surgical tools, based on information and/or instructions generated by the navigation system, for example).

The electromagnetic field emitter 120 generates an electromagnetic field in which one or more components of the system 100 are positioned or through which one or more components of the system 100 may move. In some embodiments, the electromagnetic field emitter 120 may be positioned proximate a patient. In one embodiment, the electromagnetic field emitter 120 may be positioned underneath the patient as depicted in FIG. 1A. For example, the patient may lie on a pad, pillow, or other support containing the electromagnetic field emitter 120. In other embodiments, the electromagnetic field emitter 120 may positioned elsewhere. For example, the electromagnetic field emitter 120 may be disposed underneath, within, or otherwise proximate to the imaging device 112, as depicted in FIG. 1C. In such embodiments, the known pose of the electromagnetic field emitter 120 may enable the system 100 to co-register the first localizer 144 and the second localizer 148, as discussed in further detail below. In some embodiments, the electromagnetic field emitter 120 may generate a constant electromagnetic field, while in other embodiments the electromagnetic field emitter 120 may emit a time-variant electromagnetic field.

The electromagnetic field generated and emitted by the electromagnetic field emitter 120 may interact with one or more components of the system 100, which may enable electromagnetic tracking. For example, the second localizer 148 and/or an electromagnetic tracker 156 may move through or be positioned within the electromagnetic field. The second localizer 148 and/or the electromagnetic tracker 156 may comprise one or more electromagnetic sensors that measure aspects of the electromagnetic field (e.g., magnitude of the electromagnetic filed, direction of the magnetic field, etc.). The sensor measurements may be sent to a processor of the system 100 (e.g., a processor 204 described below) that processes the measurements to determine the pose of the second localizer 148 and/or the electromagnetic tracker 156 in an electromagnetic coordinate system. Additionally or alternatively, the presence and/or movement of the second localizer 148 and/or the electromagnetic tracker 156 relative to the electromagnetic field may create measurable distortions or changes to the electromagnetic field. Such distortions may be detected by the one or more electromagnetic sensors disposed within the second localizer 148 and/or within the electromagnetic tracker 156. The processor of the system 100 may process the measured distortions to determine the pose or change in pose of the second localizer 148 and/or the electromagnetic tracker 156. A navigation system of the system 100 (e.g., a navigation system 218 discussed below) may use the information about the pose or change in pose of the second localizer 148 and/or the electromagnetic tracker 156 to, for example, track the pose of one or more anatomical elements; navigate one or more surgical instruments relative to patient anatomy, the second localizer 148, and/or the electromagnetic tracker 156; combinations thereof; and the like.

The first localizer 144 may be positioned relative to the patient, such as on the patient bed, on a surgical tool, or the like, and may be tracked optically by the navigation system. The first localizer 144 may be or comprise optical markers capable of being detected in images generated by the imaging device 112. Additionally or alternatively, the optical markers may be identifiable real-time by the imaging device 112, such as in embodiments where the imaging device 112 provide a live feed of components within the view of the imaging device 112. Based on the information (e.g., images, live stream, etc.) captured by the imaging device 112, the navigation system may identify the optical markers and use the marker location to determine the pose of the first localizer 144 in an optical coordinate system. The navigation system may then navigate one or more surgical tools (which may similarly have optical navigation markers such as the navigation markers 140A-140B) relative to the first localizer 144. In this way, the first localizer 144 may enable the navigation system to track the pose of the surgical tools being navigated relative to the first localizer 144. In other words, based on updates from the imaging device 112, the navigation system can determine a pose of the surgical tools relative to the first localizer 144.

The second localizer 148 may be positioned relative to the patient, such as relative to patient anatomy. The second localizer 148 may have a smaller footprint relative to the overall size of the first localizer 144. For example, the second localizer 148 may be or comprise an electromagnetic device implanted proximate patient anatomy. As shown in FIG. 1D, a schematic cross-section view of a vertebral section 168 according to at least one embodiment of the present disclosure may include the second localizer 148 disposed on or proximate thereto. In some embodiments, the vertebral section 168 may be or correspond to a first anatomical element 118A. The vertebral section 168 may include at least one pedicle 172, a vertebral foramen 176, a spinous process 180, a traverse process 184, nerves 188, and a vertebral body area 190. The vertebral section 168 may have the second localizer 148 disposed proximate thereto. For example, the second localizer 148 may be placed proximate one or more elements of the vertebral section 168 (e.g., the spinous process 180, the traverse process 184, etc.) to provide the system 100 with an indicator of the location of the vertebral section 168. Due to the small footprint of the second localizer 148, the second localizer 148 may be introduced to the vertebral section 168 using one or more minimally-invasive surgical techniques. For example, the second localizer 148 may be introduced to the vertebral section 168 percutaneously or using a laparoscopic or stab incision in the patient 108. In some embodiments, the second localizer 148 may be wired, while in other embodiments the second localizer 148 may be wireless.

In embodiments where the second localizer 148 comprises an electromagnetic device, the second localizer 148 may be tracked using electromagnetic tracking. For example, the second localizer 148 may interact with the electromagnetic field generated by the electromagnetic field emitter 120, such that one or more electromagnetic sensors (not shown) can measure the interaction and generate information related to the pose of the second localizer 148 relative to the electromagnetic field emitter 120. Such information may be used by a processor to determine the pose of the second localizer 148 relative to the electromagnetic field emitter 120. In some embodiments, such as when the second localizer 148 is attached to the first anatomical element 118A, the pose of the second localizer 148 may be used as or used to estimate the pose of the first anatomical element 118A for the purposes of navigating surgical tools relative to the first anatomical element 118A, for the purposes of aligning the imaging device 112 relative to the first anatomical element 118A, or for any other purpose.

The presence of both the first localizer 144 and the second localizer 148 may enable the navigation system of the system 100 to use both optical navigation and electromagnetic navigation by co-registering the first localizer 144 and the second localizer 148. The first localizer 144 may be tracked in an optical coordinate system, while the second localizer 148 may be tracked in an electromagnetic coordinate system. By determining the location of the first localizer 144 and the second localizer 148 relative to one another through co-registration, the navigation system can then determine the location of optically tracked and navigated components (e.g., surgical instruments) in the electromagnetic coordinate system as well as the location of electromagnetically tracked and navigated components (e.g., patient anatomy) in the optical coordinate system. As a result, the navigation system can navigate surgical instruments and other components relative to patient anatomy in the optical coordinate system, the electromagnetic coordinate system, a shared coordinate system, or the like.

FIG. 1E illustrates a diagram of the tracking tool 138 according to at least one embodiment of the present disclosure. The tracking tool 138 comprises the optical tracker 152 and the electromagnetic tracker 156. The optical tracker 152 may include navigation markers 160A-160D disposed in a predetermined orientation, and may be optically tracked by the navigation system based on image processing of one or more images captured by the imaging device 112. The electromagnetic tracker 156 may be tracked electromagnetically by the navigation system based on electromagnetic field measurements when the electromagnetic tracker 156 interacts with the electromagnetic field generated by the electromagnetic field emitter 120. The electromagnetic tracker 156 may be disposed in a predetermined pose relative to the optical tracker 152 or, more particularly, relative to the navigation markers 160A-160D. For example, the tracking tool 138 may be manufactured or fabricated such that the electromagnetic tracker 156 is disposed in a known pose (e.g., position and orientation) relative to the optical tracker 152. As another example, the tracking tool 138 may be placed such that navigation markers 160A-160D are disposed in predetermined locations in an electromagnetic coordinate system associated with the electromagnetic tracker 156. In such examples, the predetermined information about the pose of the electromagnetic tracker 156 relative to the optical tracker 152 and/or the coordinates of the navigation markers 160A-160D may be stored in a database and/or the electromagnetic tracker 156 (such as when the electromagnetic tracker 156 is a separate component that is detachable from the optical tracker 152) and accessed by the navigation system during the surgery or surgical procedure. Such predetermined information may also be used by the processor of the system 100 to perform co-registration of the first localizer 144 and the second localizer 148. The processor may determine the pose of the optical tracker 152 in an optical coordinate system and the pose of the electromagnetic tracker 156 in an electromagnetic coordinate system. Then, based on the predetermined information (e.g., a factory calibration of the electromagnetic tracker 156), the pose of the optical tracker 152 can be determined in the electromagnetic coordinate system and the pose of the electromagnetic tracker 156 can be determined in the optical coordinate system. Based on such information, the first localizer 144 and the second localizer 148 can be co-registered, as discussed in further detail below.

The tracking tool 138 includes an attachment mechanism 164. The attachment mechanism 164 may enable the tracking tool 138 to be attached to, mounted to, or otherwise mechanically coupled with the table 104, the patient 108, or the like. For example, the attachment mechanism 164 may enable the tracking tool 138 to be mounted to a patient bed (e.g., table 104) to enable the imaging device 112 to view and/or capture images of the tracking tool 138.

In some embodiments, the co-registration of the first localizer 144 and the second localizer 148 may be performed using the tracking tool 138. Due to the known pose of the electromagnetic tracker 156 relative to the optical tracker 152, the navigation system may be able to determine the pose of the tracking tool 138 in both an optical coordinate system and an electromagnetic coordinate system. The navigation system may then perform registration (e.g., using a processor and/or a computing device) to map optical coordinates associated with the first localizer 144 into the electromagnetic coordinate system and to map electromagnetic coordinates associated with the second localizer 148 into the optical coordinate space. To perform the co-registration using the tracking tool 138, the imaging device 112 may be caused to capture one or more images depicting the first localizer 144 and the optical tracker 152, and the navigation system may determine the pose of the first localizer 144 and the optical tracker 152 in the optical coordinate system based on the known location of the imaging device 112 when the images are captured. Similarly, the second localizer 148 and the electromagnetic tracker 156 may be disposed within the electromagnetic field generated by the electromagnetic field emitter 120. As a result, one or more electromagnetic sensors disposed within the second localizer 148 and the electromagnetic tracker 156 may generate measurements associated with various aspects of the electromagnetic field (such as the magnitude and direction of the electromagnetic field), and the navigation system may determine the pose of the first localizer 144 and the electromagnetic tracker 156 in the electromagnetic coordinate system based on the measurements from the one or more electromagnetic sensors. Based on the known or predetermined pose of the electromagnetic tracker 156 relative to the optical tracker 152, the navigation system may determine a pose of the tracking tool 138 in both the optical coordinate system and the electromagnetic coordinate system. In other words, the navigation system may determine the coordinates of the optical tracker 152 in the electromagnetic coordinate system and determine the coordinates of the electromagnetic tracker 156 in the optical coordinate system. Once the pose of the tracking tool 138 has been determined in both coordinate systems, the navigation system may then co-register (e.g., with a processor using registration) the first localizer 144 with the second localizer 148 using the pose of the tracking tool 138.

In some embodiments, the co-registration of the first localizer 144 and the second localizer 148 may be performed based on positioning an optical marker 146 relative to the electromagnetic field emitter 120. The optical marker 146 may be similar to or the same as the navigation markers 160A-160D. In other words, the optical marker 146 may be or comprise a navigation marker that can be identified in images captured by the imaging device 112. The optical marker 146 may be positioned proximate the electromagnetic field emitter 120, as depicted in FIG. 1B. For example, the optical marker 146 may be connectable to the electromagnetic field emitter 120, such that a pose of the electromagnetic field emitter 120 can be determined in an optical coordinate system. In other embodiments, the optical marker 146 may comprise a plurality of navigation markers disposed in known locations relative to the electromagnetic field emitter 120. In some embodiments, these navigation markers may be localized in the electromagnetic coordinate system during a calibration process of the electromagnetic field emitter 120. For example, the electromagnetic field emitter 120 may be provided (e.g., underneath a patient), and the optical marker 146 (or, in some embodiments, a plurality of optical markers) may be disposed on or in known locations relative to the electromagnetic field emitter 120. During the calibration process of the electromagnetic field emitter 120, the navigation system may establish the electromagnetic coordinate system based on the location of the electromagnetic field emitter 120 and determine the pose of the optical marker 146 in an electromagnetic coordinate system relative to the electromagnetic field emitter 120. The pose of the optical marker 146 may then also be determined in an optical coordinate system (e.g., based on images captured by the imaging device 112). Since the pose of the optical marker 146 is known in both the optical coordinate system and the electromagnetic coordinate system, the navigation system may co-register (e.g., with a processor using registration) the first localizer 144 and the second localizer 148 once the first localizer 144 is localized in the optical coordinate system and the second localizer 148 is localized in the electromagnetic coordinate system.

In some embodiments, the first localizer 144 and the second localizer 148 may be co-registered based on electromagnetically tracking the imaging device 112. In such embodiments, the electromagnetic field emitter 120 may be disposed inside, underneath, or proximate to the imaging device 112, as depicted in FIG. 1C. The electromagnetic field emitter 120 may emit the electromagnetic field that can be used to localize the second localizer 148 in an electromagnetic coordinate system. Additionally, the imaging device 112 may capture one or more images depicting the first localizer 144, allowing the first localizer 144 to be localized in an optical coordinate system. Since the electromagnetic field emitter 120 is disposed within the imaging device 112, the pose of the electromagnetic field emitter 120 can be determined in the optical coordinate system, while the pose of the imaging device 112 can be determined in the electromagnetic coordinate system. The navigation system may then co-register (e.g., with a processor using registration) the first localizer 144 with the second localizer 148.

Once the first localizer 144 and the second localizer 148 are co-registered, the navigation system may navigate one or more surgical tools relative to patient anatomy based on the tracking of the first localizer 144 and the second localizer 148. In other words, the imaging device 112 may continue to capture image data of the first localizer 144 and the electromagnetic sensors may continue to capture measurements associated with the electromagnetic field to track the second localizer 148, with any change in the pose thereof respectively captured by the imaging device 112 or the electromagnetic sensors. The navigation system may use the first localizer 144 to track movement of surgical tools or other surgical components (e.g., the imaging device 112) in the optical system, and may use the second localizer 148 to track movement of the patient anatomy proximate the second localizer 148. Due to the small footprint of the second localizer 148, the navigation system may beneficially track patient anatomy without using an optical component that may be otherwise difficult to attach to patient anatomy. Moreover, the use of the second localizer 148 removes the need to have patient anatomy tracked by the second localizer 148 within the line-of-sight of the imaging device 112 or other optical-based component.

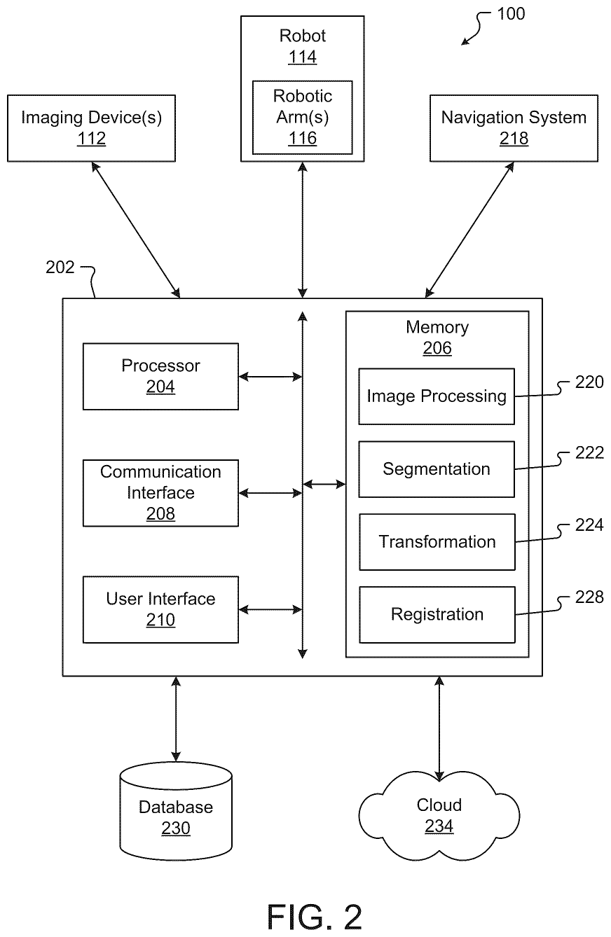

Turning next to FIG. 2, a block diagram of additional aspects of the system 100 according to at least one embodiment of the present disclosure is shown. The additional aspects of the system 100 include a computing device 202, the navigation system 218, a database 230, and a cloud or other network 234. As shown in FIG. 2, the imaging device 112, the robot 114, and the robotic arm 116 may be in communication with the computing device 202 (and components thereof), the navigation system 218, the database 230, and/or the cloud 234.

The computing device 202 comprises a processor 204, a memory 206, a communication interface 208, and a user interface 210. Computing devices according to other embodiments of the present disclosure may comprise more or fewer components than the computing device 202.

The processor 204 of the computing device 202 may be any processor described herein or any similar processor. The processor 204 may be configured to execute instructions stored in the memory 206, which instructions may cause the processor 204 to carry out one or more computing steps utilizing or based on data received from the imaging device 112, the robot 114, the navigation system 218, the database 230, and/or the cloud 234.

The memory 206 may be or comprise RAM, DRAM, SDRAM, other solid-state memory, any memory described herein, or any other tangible, non-transitory memory for storing computer-readable data and/or instructions. The memory 206 may store information or data useful for completing, for example, any step of the methods 300, 400, 500, and/or 600 described herein, or of any other methods. The memory 206 may store, for example, instructions and/or machine learning models that support one or more functions of the robot 114. For instance, the memory 206 may store content (e.g., instructions and/or machine learning models) that, when executed by the processor 204, enable image processing 220, segmentation 222, transformation 224, and/or registration 228. Such content, if provided as in instruction, may, in some embodiments, be organized into one or more applications, modules, packages, layers, or engines. Alternatively or additionally, the memory 206 may store other types of content or data (e.g., machine learning models, artificial neural networks, deep neural networks, etc.) that can be processed by the processor 204 to carry out the various method and features described herein. Thus, although various contents of memory 206 may be described as instructions, it should be appreciated that functionality described herein can be achieved through use of instructions, algorithms, and/or machine learning models. The data, algorithms, and/or instructions may cause the processor 204 to manipulate data stored in the memory 206 and/or received from or via the imaging device 112, the robot 114, the database 230, and/or the cloud 234.

The computing device 202 may also comprise a communication interface 208. The communication interface 208 may be used for receiving image data or other information from an external source (such as the imaging device 112, the robot 114, the navigation system 218, the database 230, the cloud 234, and/or any other system or component not part of the system 100), and/or for transmitting instructions, images, or other information to an external system or device (e.g., another computing device 202, the imaging device 112, the robot 114, the navigation system 218, the database 230, the cloud 234, and/or any other system or component not part of the system 100). The communication interface 208 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 208 may be useful for enabling the computing device 202 to communicate with one or more other processors 204 or computing devices 202, whether to reduce the time needed to accomplish a computing-intensive task or for any other reason.

The computing device 202 may also comprise one or more user interfaces 210. The user interface 210 may be or comprise a keyboard, mouse, trackball, monitor, television, screen, touchscreen, and/or any other device for receiving information from a user and/or for providing information to a user. The user interface 210 may be used, for example, to receive a user selection or other user input regarding any step of any method described herein. Notwithstanding the foregoing, any required input for any step of any method described herein may be generated automatically by the system 100 (e.g., by the processor 204 or another component of the system 100) or received by the system 100 from a source external to the system 100. In some embodiments, the user interface 210 may be useful to allow a surgeon or other user to modify instructions to be executed by the processor 204 according to one or more embodiments of the present disclosure, and/or to modify or adjust a setting of other information displayed on the user interface 210 or corresponding thereto.

Although the user interface 210 is shown as part of the computing device 202, in some embodiments, the computing device 202 may utilize a user interface 210 that is housed separately from one or more remaining components of the computing device 202. In some embodiments, the user interface 210 may be located proximate one or more other components of the computing device 202, while in other embodiments, the user interface 210 may be located remotely from one or more other components of the computing device 202.

The navigation system 218 may provide navigation for a surgeon and/or a surgical robot during an operation. The navigation system 218 may be any now-known or future-developed navigation system, including, for example, the Medtronic StealthStation™ S8 surgical navigation system or any successor thereof. The navigation system 218 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 218 may comprise one or more electromagnetic sensors. In various embodiments, the navigation system 218 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 218 may include a display for displaying one or more images from an external source (e.g., the computing device 202, imaging device 112, or other source) or for displaying an image and/or video stream from the one or more cameras or other sensors of the navigation system 218. In some embodiments, the system 100 can operate without the use of the navigation system 218. The navigation system 218 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

As noted above, the navigation system 218 navigates one or more surgical instruments based on the pose of the first localizer 144 and/or the second localizer 148. Once the first localizer 144 and the second localizer 148 are co-registered, such as by using the tracking tool 138, using an optical marker 146 and the electromagnetic field emitter 120, and/or using the electromagnetic field emitter 120 disposed within the imaging device 112, the navigation system 218 may use the first localizer 144 to navigate surgical tools, the imaging device 112, or other components of the system 100. Additionally or alternatively, the navigation system 218 may use the second localizer 148 to determine the pose of the patient anatomy (e.g., anatomical elements 118A-118N) and, based on the co-registration, navigate the surgical tools relative to the patient anatomy.

The database 230 may store information that correlates one coordinate system to another (e.g., one or more robotic coordinate systems to a patient coordinate system and/or to a navigation coordinate system). The database 230 may additionally or alternatively store, for example, one or more surgical plans (including, for example, pose information about a target and/or image information about a patient's anatomy at and/or proximate the surgical site, for use by the robot 114, the navigation system 218, and/or a user of the computing device 202 or of the system 100); one or more images useful in connection with a surgery to be completed by or with the assistance of one or more other components of the system 100; and/or any other useful information. The database 230 may be configured to provide any such information to the computing device 202 or to any other device of the system 100 or external to the system 100, whether directly or via the cloud 234. In some embodiments, the database 230 may be or comprise part of a hospital image storage system, such as a picture archiving and communication system (PACS), a health information system (HIS), and/or another system for collecting, storing, managing, and/or transmitting electronic medical records including image data.

The cloud 234 may be or represent the Internet or any other wide area network. The computing device 202 may be connected to the cloud 234 via the communication interface 208, using a wired connection, a wireless connection, or both. In some embodiments, the computing device 202 may communicate with the database 230 and/or an external device (e.g., a computing device) via the cloud 234.

The system 100 or similar systems may be used, for example, to carry out one or more aspects of any of the methods 300, 400, 500, and/or 600 described herein. The system 100 or similar systems may also be used for other purposes.

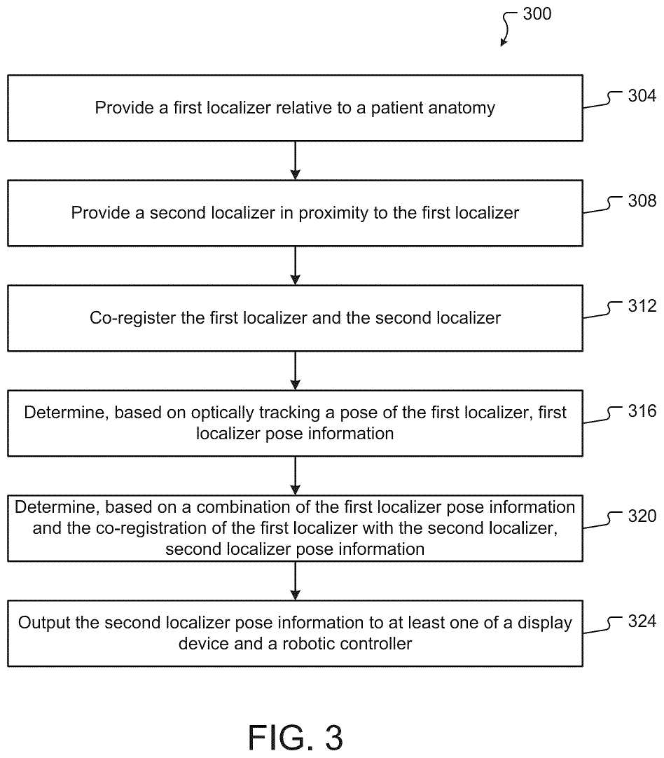

FIG. 3 depicts a method 300 that may be used, for example, to co-register localizers to facilitate surgical navigation.

The method 300 (and/or one or more steps thereof) may be carried out or otherwise performed, for example, by at least one processor. The at least one processor may be the same as or similar to the processor(s) 204 of the computing device 202 described above. The at least one processor may be part of a robot (such as a robot 114) or part of a navigation system (such as a navigation system 218). A processor other than any processor described herein may also be used to execute the method 300. The at least one processor may perform the method 300 by executing elements stored in a memory such as the memory 206. The elements stored in memory and executed by the processor may cause the processor to execute one or more steps of a function as shown in method 300. One or more portions of a method 300 may be performed by the processor executing any of the contents of memory, such as an image processing 220, a segmentation 222, a transformation 224, and/or a registration 228.

The method 300 comprises providing a first localizer relative to a patient anatomy (step 304). The first localizer may be similar to or the same as the first localizer 144. The first localizer 144 may be an optical marker, such as a marker capable of being identified in an optical coordinate system based on images captured by the imaging device 112, and may be used to track the pose of one or more surgical tools relative to a patient (e.g., patient 108).

The method 300 also comprises providing a second localizer in proximity to the first localizer (step 308). The second localizer may be similar to or the same as the second localizer 148. The second localizer 148 may be an electromagnetic marker, such as a marker capable of being identified based on electromagnetic field distortion measurements captured by one or more electromagnetic sensors. The second localizer 148 may be used to track the pose of patient anatomy (e.g., anatomical elements 118A-118N).

The method 300 also comprises co-registering the first localizer and the second localizer (step 312). The co-registering of the first localizer and the second localizer may include determining coordinates of the first localizer in an electromagnetic coordinate system and coordinates of the second localizer in an optical coordinate system. In some embodiments, the co-registering may include using a tracking tool 138 with both an optical tracker 152 and an electromagnetic tracker 156 to perform the co-registration. In some embodiments, the co-registering may be based on the known pose of one or more navigation markers (e.g., optical marker 146) in an electromagnetic coordinate system determined, for example, during a calibration process of the electromagnetic field emitter 120. In some embodiments, the co-registering may be based on an electromagnetically tracked camera, such as when the electromagnetic field emitter 120 is disposed within the imaging device 112.

The method 300 also comprises determining, based on optically tracking a pose of the first localizer, first localizer pose information (step 316). The first localizer pose information may comprise information about the location and orientation of the first localizer 144 in an optical coordinate system, and may be based on one or more images captured by the imaging device 112. The navigation system 218 may use the processor 204 to perform image processing on the images captured by the imaging device 112, and segment the images (e.g., using segmentation 222) to segment the first localizer (and any other navigation markers) depicted in the images. Based on the identified first localizer and the known pose of the imaging device 112 when the images were captured, the navigation system 218 may use one or more transformations 224 to determine pose of the first localizer 144.

The method 300 also comprises determining, based on a combination of the first localizer pose information and the co-registration of the first localizer with the second localizer, second localizer pose information (step 320). The second localizer pose information may comprise a pose (e.g., a location and orientation) of the second localizer 148. In some embodiments, the navigation system 218 may map (e.g., using registration 228) the location of the first localizer 144 into an electromagnetic coordinate system associated with the second localizer, and then determine the pose of the second localizer 148 based on the co-registration of the first localizer 144 and the second localizer 148 determined, for example, in the step 312.

The method 300 also comprises outputting the second localizer pose information to at least one of a display device and a robotic controller (step 324). In some embodiments, the display device may be or comprise the user interface 210 and the robotic controller may be or comprise the navigation system 218. Based on the second localizer pose information, the navigation system 218 may navigate one or more surgical tools relative to the second localizer 148. In some embodiments, the second localizer 148 may represent the location of patient anatomy, such as when the second localizer 148 is disposed on the patient anatomy.

The present disclosure encompasses embodiments of the method 300 that comprise more or fewer steps than those described above, and/or one or more steps that are different than the steps described above.

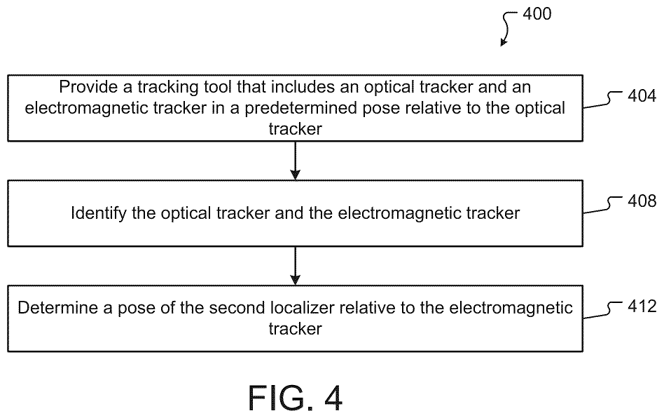

FIG. 4 depicts a method 400 that may be used, for example, to co-register localizers using a tracking tool.

The method 400 (and/or one or more steps thereof) may be carried out or otherwise performed, for example, by at least one processor. The at least one processor may be the same as or similar to the processor(s) 204 of the computing device 202 described above. The at least one processor may be part of a robot (such as a robot 114) or part of a navigation system (such as a navigation system 218). A processor other than any processor described herein may also be used to execute the method 400. The at least one processor may perform the method 400 by executing elements stored in a memory such as the memory 206. The elements stored in the memory and executed by the processor may cause the processor to execute one or more steps of a function as shown in method 400. One or more portions of a method 400 may be performed by the processor executing any of the contents of memory, such as an image processing 220, a segmentation 222, a transformation 224, and/or a registration 228.

The method 400 comprises providing a tracking tool that includes an optical tracker and an electromagnetic tracker in a predetermined pose relative to the optical tracker (step 404). The tracking tool may be similar to or the same as the tracking tool 138, while the optical tracker and the electromagnetic tracker may be similar to or the same as the optical tracker 152 and the electromagnetic tracker 156, respectively. In some embodiments, the tracking tool 138 may be disposed on or next to a patient or a patient bed. The electromagnetic tracker 156 may be disposed in a predetermined pose relative to the optical tracker 152 due to, for example, the fabrication or manufacturing of the tracking tool 138. In other embodiments, the electromagnetic tracker 156 may be detachable from the tracking tool 138, and may have a predetermined location (e.g., a slot in the optical tracker 152) into which the electromagnetic tracker 156 can be inserted, such that the electromagnetic tracker 156 is in a known or predetermined pose relative to the optical tracker 152 when the electromagnetic tracker 156 is connected to the optical tracker 152. In some embodiments, information relating to the pose of the electromagnetic tracker 156 relative to the optical tracker 152 (or vice versa) may be stored in the database 230.

The method 400 also comprises identifying the optical tracker and the electromagnetic tracker (step 408). The optical tracker 152 may be identified and localized using one or more optical components of the system 100, such as by capturing one or more images depicting the optical tracker 152. The one or more images may then be processed (e.g., using image processing 220) and segmented (e.g., using segmentation 222) to identify the optical tracker 152. The electromagnetic tracker 156 may be identified using measurements captured by one or more electromagnetic sensors based on interactions between the electromagnetic tracker 156 and an electromagnetic field generated by the electromagnetic field emitter 120.

The method 400 also comprises determining a pose of the second localizer relative to the electromagnetic tracker (step 412). The second localizer 148 may also interact with the electromagnetic field generated by the electromagnetic field emitter 120, and the one or more electromagnetic sensors may capture such interactions. The sensor measurements may be sent to the processor 204, which may process the measurements to determine the location of the second localizer 148 in the electromagnetic coordinate system. Since both the second localizer 148 and the electromagnetic tracker 156 interact with the electromagnetic field, the processor 204 can determine the pose of the second localizer 148 relative to the electromagnetic tracker 156 (e.g., based on one or more transformations 224 of the measurements provided by the electromagnetic sensors). In some embodiments, after determining the pose of the second localizer 148 relative to the electromagnetic tracker 156, the processor 204 may use the predetermined pose of the electromagnetic tracker 156 relative to the optical tracker 152 to determine the pose of the second localizer 148 in an optical coordinate system, which may further enable the processor 204 to co-register the second localizer 148 with the first localizer 144.

The present disclosure encompasses embodiments of the method 400 that comprise more or fewer steps than those described above, and/or one or more steps that are different than the steps described above.

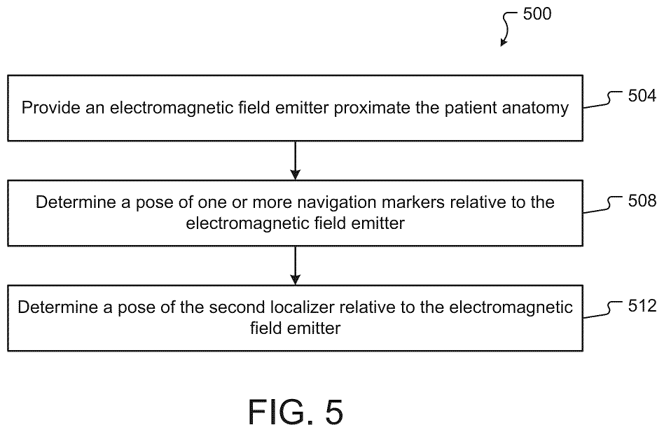

FIG. 5 depicts a method 500 that may be used, for example, to co-register localizers using navigation markers and an electromagnetic field emitter.

The method 500 (and/or one or more steps thereof) may be carried out or otherwise performed, for example, by at least one processor. The at least one processor may be the same as or similar to the processor(s) 204 of the computing device 202 described above. The at least one processor may be part of a robot (such as a robot 114) or part of a navigation system (such as a navigation system 218). A processor other than any processor described herein may also be used to execute the method 500. The at least one processor may perform the method 500 by executing elements stored in a memory such as the memory 206. The elements stored in the memory and executed by the processor may cause the processor to execute one or more steps of a function as shown in method 500. One or more portions of a method 500 may be performed by the processor executing any of the contents of memory, such as an image processing 220, a segmentation 222, a transformation 224, and/or a registration 228.

The method 500 comprises providing an electromagnetic field emitter proximate the patient anatomy (step 504). The electromagnetic field emitter may be similar to or the same as the electromagnetic field emitter 120. The patient anatomy may be similar to or the same as the anatomical elements 118A-118N. In some embodiments, the electromagnetic field emitter 120 may be disposed on the table 104 before the patient 108 rests on the table. In such embodiments, the electromagnetic field emitter 120 may be disposed underneath the patient, such that the electromagnetic field emitter 120 can generate an electromagnetic field in an area proximate the patient 108. In some embodiments, the electromagnetic field may be constant in time and/or intensity, while in other embodiments the electromagnetic field may vary in intensity and/or direction with time. The electromagnetic field may interact with the second localizer 148, the electromagnetic tracker 156, and/or other electromagnetic components (e.g., electromagnetic sensors).

The method 500 also comprises determining a pose of one or more navigation markers relative to the electromagnetic field emitter (step 508). Once the electromagnetic field emitter 120 has been disposed underneath or near the patient 108, one or more navigation markers (e.g., an optical marker 146) may be placed in known locations proximate the electromagnetic field emitter 120. In some embodiments, the locations of the one or more navigation markers may be known in the electromagnetic coordinate system. In some embodiments, the locations of the one or more navigation markers may be determined when the electromagnetic field emitter 120 is calibrated.

The method 500 also comprises determining a pose of the second localizer relative to the electromagnetic field emitter (step 512). Once the electromagnetic field emitter 120 has been calibrated, measurements from one or more electromagnetic sensors may be used (e.g., by the processor 204) to determine the pose of the second localizer 148 relative to the electromagnetic field emitter 120. The measurements from the one or more electromagnetic sensors may comprise information about the aspects of the electromagnetic field emitted by the electromagnetic field emitter 120 that are measured at the second localizer 148, allowing the processor 204 (e.g., using transformation 224) to determine the pose (e.g., position and orientation) of the second localizer 148 relative to the electromagnetic field emitter 120. In some embodiments, after determining the pose of the second localizer 148 relative to the electromagnetic field emitter 120, the processor 204 may use the determined pose of the optical marker 146 relative to the electromagnetic field emitter 120 to determine the pose of the second localizer 148 in an optical coordinate system (since the pose of the one or more navigation markers such as the optical marker 146 is also known in an optical coordinate system), which may further enable the processor 204 to co-register the second localizer 148 with the first localizer 144.

The present disclosure encompasses embodiments of the method 500 that comprise more or fewer steps than those described above, and/or one or more steps that are different than the steps described above.

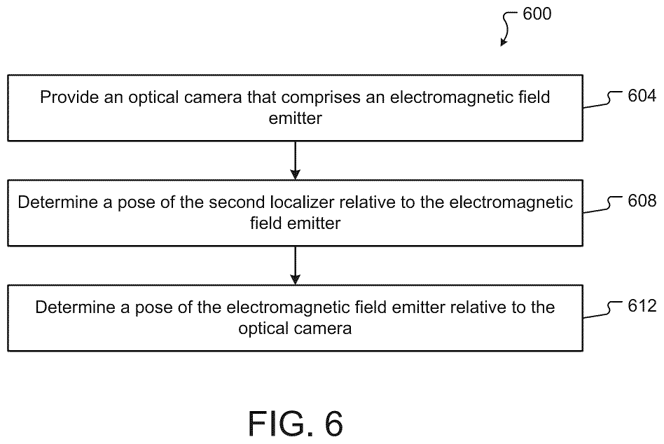

FIG. 6 depicts a method 600 that may be used, for example, to co-register localizers using an optical camera with an electromagnetic field emitter.

The method 600 (and/or one or more steps thereof) may be carried out or otherwise performed, for example, by at least one processor. The at least one processor may be the same as or similar to the processor(s) 204 of the computing device 202 described above. The at least one processor may be part of a robot (such as a robot 114) or part of a navigation system (such as a navigation system 218). A processor other than any processor described herein may also be used to execute the method 600. The at least one processor may perform the method 600 by executing elements stored in a memory such as the memory 206. The elements stored in the memory and executed by the processor may cause the processor to execute one or more steps of a function as shown in method 600. One or more portions of a method 600 may be performed by the processor executing any of the contents of memory, such as an image processing 220, a segmentation 222, a transformation 224, and/or a registration 228.

The method 600 comprises providing an optical camera that comprises an electromagnetic field emitter (step 604). In some embodiments, the optical camera may be similar to or the same as the imaging device 112, and the electromagnetic field emitter may be similar to or the same as the electromagnetic field emitter 120. The electromagnetic field emitter 120 may be disposed at least partially within the imaging device 112, such that the electromagnetic field emitter 120 moves with the imaging device 112, such as when the robot 114 repositions the imaging device 112. In some embodiments, the imaging device 112 may be positioned in proximity to the patient 108, such that the electromagnetic field emitter 120 can emit an electromagnetic field that interacts with the second localizer 148.

The method 600 also comprises determining a pose of the second localizer relative to the electromagnetic field emitter (step 608). In some embodiments, the second localizer may be similar to or the same as the second localizer 148. As previously discussed, the second localizer 148 may comprise one or more electromagnetic sensors that measure aspects of the electromagnetic field generated by the electromagnetic field emitter 120. The processor 204 may use such measurements to determine the pose of the second localizer 148 relative to the electromagnetic field emitter 120.

The method 600 also comprises determining a pose of the electromagnetic field emitter relative to the optical camera (step 612). The electromagnetic field emitter 120 can be at least partially disposed within the imaging device 112, which may mean that the coordinates of the electromagnetic field emitter 120 in the electromagnetic coordinate system may be the same as the imaging device 112. The navigation system 218 may use the processor 204 to determine the pose of the imaging device 112 in the optical coordinate system (e.g., based on the optical tracking of the first localizer 144 by the imaging device 112). In some embodiments, the navigation system 218 may then use the processor 204 to co-register the first localizer 144 with the second localizer 148.

The present disclosure encompasses embodiments of the method 600 that comprise more or fewer steps than those described above, and/or one or more steps that are different than the steps described above.

As noted above, the present disclosure encompasses methods with fewer than all of the steps identified in FIGS. 3, 4, 5, and 6 (and the corresponding description of the methods 300, 400, 500, and 600), as well as methods that include additional steps beyond those identified in FIGS. 3, 4, 5, and 6 (and the corresponding description of the methods 300, 400, 500, and 600). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The techniques of this disclosure may also be described in the following examples.

Example 1: A system (100), comprising:
a first localizer (144);
a second localizer (148) positionable proximate the first localizer (144);
a processor (204); and
a memory (206) storing data thereon that, when processed by the processor (204), cause the processor (204) to:
co-register the first localizer (144) and the second localizer (148);
determine, based on tracking a pose of the first localizer (144), first localizer pose information;
determine, based on a combination of the first localizer pose information and the co-registration of the first localizer (144) with the second localizer (148), second localizer pose information; and
output the second localizer pose information to at least one of a display device (210) and a robotic controller (218).

Example 2: The system of example 1, wherein the first localizer (144) is positioned relative to an anatomical element (118A, 118B, 118N) and disposed on at least one of a surgical tool and a bed mount.

Example 3: The system of examples 1 or 2, wherein the second localizer (148) comprises an electromagnetic device.

Example 4: The system of any of examples 2 to 3, wherein the anatomical element (118A, 118B, 118N) comprises a vertebra, and wherein the second localizer (148) is disposable on the vertebra.

Example 5: The system of any of examples 1 to 4, wherein the first localizer (144) and the second localizer (148) are co-registered by:
identifying an optical tracker (152) and an electromagnetic tracker (156) disposed in a pose relative to the optical tracker (152).

Example 6: The system of any of examples 1 to 4, wherein the first localizer (144) and the second localizer (148) are co-registered by:
determining a pose of one or more optically tracked navigation markers (146) relative to an electromagnetic field emitter (120).

Example 7: The system of any of examples 1 to 4, wherein the first localizer (144) and the second localizer (148) are co-registered by:
determining a pose of the second localizer (148) relative to an electromagnetic field emitter (120); and
determining a pose of the electromagnetic field emitter (120) relative to an optical camera (112) that optically tracks the pose of the first localizer (144).

Example 8: The system of example 1, wherein one or more surgical instruments are navigated based at least partially on the second localizer pose information.

Example 9: A system (100), comprising:
a processor (204); and
a memory (206) storing data thereon that, when processed by the processor (204), cause the processor (204) to:
co-register a first localizer (144) and a second localizer (148), the first localizer positionable relative to a patient anatomy (118A, 118B, 118N) and the second localizer (148) positionable proximate the first localizer (144);
determine, based on information from an optical camera (112) that tracks a pose of the first localizer (144), first localizer pose information;
determine, based on a combination of the first localizer pose information and the co-registration of the first localizer (144) with the second localizer (148), second localizer pose information; and
output the second localizer pose information to at least one of a display device (210) and a robotic controller (218).

Example 10: The system of example 9, wherein the co-registering the first localizer (144) and the second localizer (148) comprises:
identifying a tracking tool (138) that comprises one or more one or more optical navigation markers (160A-160D) and an electromagnetic tracker (156) disposed in a pose relative to the one or more optical navigation markers (160A-160D).

Example 11: The system of example 9, wherein the co-registering the first localizer (144) and the second localizer (148) comprises:
determining a pose of one or more optical navigation markers (146) relative to an electromagnetic field emitter (120).

Example 12: The system of example 9, wherein the co-registering the first localizer (144) and the second localizer (148) comprises:
determining a pose of the second localizer (148) relative to an electromagnetic field emitter (120); and
determining a pose of the electromagnetic field emitter (120) relative to the optical camera (112).

Example 13: The system of any of examples 9 to 12, wherein the robotic controller (218) navigates one or more surgical instruments based at least partially on the second localizer pose information.

Example 14: A method, comprising:
providing a first localizer (144) relative to a patient anatomy (118A, 118B, 118N);
providing a second localizer (148) in proximity to the first localizer (144);
co-registering the first localizer (144) and the second localizer (148);
determining, based on tracking a pose of the first localizer (144), first localizer pose information;
determining, based on a combination of the first localizer pose information and the co-registration of the first localizer (144) with the second localizer (148), second localizer pose information; and
outputting the second localizer pose information to at least one of a display device (210) and a robotic controller (218).

Example 15: The method of example 14, wherein the co-registering the first localizer (144) and the second localizer (148) comprises:
providing a tracking tool (138) that includes an optical tracker (152) and an electromagnetic tracker (156) in a predetermined pose relative to the optical tracker (152).

Various examples of the disclosure have been described. These and other examples are within the scope of the following claims.

What is claimed is:

1. A method, comprising:
providing a first localizer relative to a patient anatomy;
providing a second localizer in proximity to the first localizer;
co-registering the first localizer and the second localizer;
determining, based on tracking a pose of the first localizer, first localizer pose information;
determining, based on a combination of the first localizer pose information and the co-registration of the first localizer with the second localizer, second localizer pose information; and
outputting the second localizer pose information to at least one of a display device and a robotic controller.

2. The method of claim 1, wherein one or more surgical instruments are navigated based at least partially on the second localizer pose information.

3. The method of claim 1, wherein the co-registering the first localizer and the second localizer comprises:
providing a tracking tool that includes an optical tracker and an electromagnetic tracker in a predetermined pose relative to the optical tracker.

4. The method of claim 3, wherein the optical tracker comprises a plurality of navigation markers, and wherein the method further comprises:
determining a pose of the second localizer relative to the electromagnetic tracker.

5. The method of claim 3, wherein the tracking tool is provided on at least one of a patient and a patient bed.

6. The method of claim 1, wherein the co-registering the first localizer and the second localizer comprises:
providing an electromagnetic field emitter proximate the patient anatomy; and
determining a pose of one or more navigation markers relative to the electromagnetic field emitter.

7. The method of claim 6, wherein the one or more navigation markers are tracked optically.

8. The method of claim 1, further comprising:
providing an optical camera that comprises an electromagnetic field emitter and that tracks the pose of the first localizer; and
determining a pose of the second localizer relative to the electromagnetic field emitter.

9. A system, comprising:
a first localizer;
a second localizer positionable proximate the first localizer;
a processor; and
a memory storing data thereon that, when processed by the processor, cause the processor to:
co-register the first localizer and the second localizer;
determine, based on tracking a pose of the first localizer, first localizer pose information;
determine, based on a combination of the first localizer pose information and the co-registration of the first localizer with the second localizer, second localizer pose information; and
output the second localizer pose information to at least one of a display device and a robotic controller.

10. The system of claim 9, wherein the first localizer is positioned relative to an anatomical element and disposed on at least one of a surgical tool and a bed mount.

11. The system of claim 10, wherein the second localizer comprises an electromagnetic device.

12. The system of claim 11, wherein the anatomical element comprises a vertebra, and wherein the second localizer is disposable on the vertebra.

13. The system of claim 9, wherein the first localizer and the second localizer are co-registered by:
identifying an optical tracker and an electromagnetic tracker disposed in a pose relative to the optical tracker.

14. The system of claim 9, wherein the first localizer and the second localizer are co-registered by:
determining a pose of one or more optically tracked navigation markers relative to an electromagnetic field emitter.

15. The system of claim 9, wherein the first localizer and the second localizer are co-registered by:
determining a pose of the second localizer relative to an electromagnetic field emitter; and
determining a pose of the electromagnetic field emitter relative to an optical camera that optically tracks the pose of the first localizer.

16. A system, comprising:
a processor; and
a memory storing data thereon that, when processed by the processor, cause the processor to:
co-register a first localizer and a second localizer, the first localizer positionable relative to a patient anatomy and the second localizer positionable proximate the first localizer;
determine, based on information from an optical camera that tracks a pose of the first localizer, first localizer pose information;
determine, based on a combination of the first localizer pose information and the co-registration of the first localizer with the second localizer, second localizer pose information; and
output the second localizer pose information to at least one of a display device and a robotic controller.

17. The system of claim 16, wherein the co-registering the first localizer and the second localizer comprises:
identifying a tracking tool that comprises one or more one or more optical navigation markers and an electromagnetic tracker disposed in a pose relative to the one or more optical navigation markers.

18. The system of claim 16, wherein the co-registering the first localizer and the second localizer comprises:
determining a pose of one or more optical navigation markers relative to an electromagnetic field emitter.

19. The system of claim 16, wherein the co-registering the first localizer and the second localizer comprises:
determining a pose of the second localizer relative to an electromagnetic field emitter; and
determining a pose of the electromagnetic field emitter relative to the optical camera.

20. The system of claim 16, wherein the robotic controller navigates one or more surgical instruments based at least partially on the second localizer pose information.
